(19) United States
(12) Patent Application Publication
(10) Pub. No.: US 2012/0095555 A1
(43) Pub. Date: Apr. 19, 2012

(54) MEDICAL IMPLANT, THIN FILM THEREON, AND METHOD FOR MANUFACTURING THE SAME

(75) Inventors: Pei-Huan HSIEH, New Taipei City (TW); Min-Hua Chen, Kaohsiung City (TW)

(73) Assignee: METAL INDUSTRIES RESEARCH&DEVELOPMENT CENTRE, Kaohsiung (TW)

(21) Appl. No.: 13/268,085

(22) Filed: Oct. 7, 2011

(30) Foreign Application Priority Data
Oct. 18, 2010 (TW) 099135397

(51) Int. Cl. A61F 2/02; C25D 3/02; C25D 9/04; C25D 7/00; C25D 5/00

(52) U.S. Cl. 623/11.11; 205/80; 205/318; 205/333; 205/316; 205/322

(57) ABSTRACT

A thin film of a medical implant includes a surface, a plurality of walls and a plurality of paths. The walls are disposed on the surface, and formed to shapes of arc. The paths are disposed on the surface, wherein each of the paths is located among the walls. The walls and paths have a plurality of holes. According to the thin film of the present disclosure, the walls are formed to shapes of arc, and have no acute anger, whereby the biological cells can helpfully grow and attach on the thin film quickly. Furthermore, the thin film has the holes, which provide cell tissue, such as pseudopod, tentacle, etc. of the biological cells to grow and attach therein, whereby the biological cyto-affinity of the thin film can be increased so as to increase the biological cyto-compatibility of the medical implant.

### CROSS-REFERENCE TO RELATED APPLICATION

This application claims the benefit of Taiwan Patent Application No. 099135397, filed on Oct. 18, 2010, which is hereby incorporated by reference for all purposes as if fully set forth herein.

### BACKGROUND

1. Field of Invention

The present disclosure relates to a medical implant, and more particularly to a medical implant, a thin film thereon and a method for manufacturing the same, wherein the medical implant can increase a biological cyto-affinity.

2. Related Art

As the national income is continuously increased, the population is aged and advanced medical technology is imported, people think that the necessity of medical treatment and health protection is important gradually. Specially, human medical implant is developed, such as tooth and bone implant. Currently, the population is gradually aged in the society. Human joint, bone and tooth will be gradually degenerated after many years, and thus human will have an inconvenient life. Artificial substitute or mounting element must be implanted, whereby the degenerated joint, bone and tooth can be replaced with or mounted by the artificial substitute, such as artificial joint, artificial bone screw and artificial tooth so as to maintain the functions. Thus, people gradually pay much attention to medical implant, and particularly to a biological cyto-compatibility and a biological cyto-affinity of a medical implant.

Currently, medical experiments prove that biological cells grow uneasily on a smooth surface of the medical implant. In order to easily grow the biological cells on the surface of the medical implant, the surface of current medical implant must be formed to a rough surface by a surface treatment process, thereby increasing the biological cyto-compatibility and the biological cyto-affinity of the medical implant. In the past, the surface of current medical implant is treated by a mechanic machining process, but it is necessary to have long time for finishing bone conformity so as not to meet the current medical requirement. Currently, the surface of current medical implant is treated by an oxidizing acid, such as sulfuric acid, hydrochloric acid, etc. Furthermore, it is proved that the effect of the surface treated by the oxidizing acid is better than that treated by the mechanic machining process. However, according to different concentration, temperature, time and mixing method of the sulfuric acid and the hydrochloric acid, the acid etching is different to cause different surface condition. The surface roughness generated by the acid etching is still unstable and undesirable. Thus, the biological cyto-compatibility and the biological cyto-affinity of the medical implant cannot be increased effectively.

Currently, a method for manufacturing a medical implant which achieves the objective of the biological cyto-compatibility and the biological cyto-affinity mainly includes physical treatment processes, such as a SLA (sand-blasted large-grit acid-etched) process, a thermal spraying process, etc., and chemical treatment processes, such as a etching process, a thermal oxidization process, etc. so as to achieves the objective of the biological cyto-compatibility and the biological cyto-affinity. Although the method for manufacturing a medical implant mainly uses the SLA process, the SLA process uses a chemical agent having more environmental pollution so as not to meet the requirement of environmental protection and have clean cost. Currently, new surface treatment processes of the medical implant are continually disclosed on the cited references, such as U.S. Pat. No. 5,603,338, U.S. Pat. No. 5,456,723 and Taiwan patent no. 1244958. However, the effect of the treated surface is still limited, and this rough surface cannot provide biological cell to fast and easily grow thereon. Thus, the fast and easily biological cyto-compatibility and cyto-affinity of the medical implant cannot be increased obviously.

### SUMMARY

Accordingly, the present disclosure provides a medical implant, a thin film thereon and a method for manufacturing the same, wherein the medical implant can improve the fast and easily biological cyto-compatibility and cyto-affinity of the conventional thin film thereon, decrease the manufacture cost, increase stable quality, and decrease the environmental pollution so as to solve the above-mentioned problem in the prior art.

The present disclosure provides a medical implant and a thin film thereof. The thin film includes a plurality of walls and a plurality of paths. The walls are formed to shapes of arc, and have no acute anger. The walls and paths have a plurality of holes, whereby the biological cells can helpfully grow and attach on the thin film quickly so as to increase biological cyto-compatibility and cyto-affinity of the medical implant.

The present disclosure provides a method for manufacturing a thin film of a medical implant, the method adapted to form the thin film on the surface of the medical implant body by the chemical electrolysis machining process, whereby the manufacture cost can be decreased, the quality is stable, and the environmental pollution can be decreased.

A thin film of a medical implant of the present disclosure includes a surface, a plurality of walls and a plurality of paths. The walls are disposed on the surface, and formed to shapes of arc. The paths are disposed on the surface, wherein the path is located among the walls. All of the walls and paths have a plurality of holes. According to the thin film of the present disclosure, the walls are formed to shapes of arc, and have no acute anger, whereby the biological cells can helpfully grow and attach on the thin film quickly. Furthermore, the thin film has the holes, which provide cell tissue, such as pseudopod, tentacle, etc. of the biological cells to grow and attach therein, whereby the biological cyto-affinity of the thin film can be increased so as to increase the biological cyto-compatibility of the medical implant.

A method for manufacturing the thin film of the present disclosure includes the following steps. A tank having an electrolyte and an electrode is provided. The medical implant body is put in the tank and dipped into the electrolyte. An anode and a cathode of a power source unit are electrically connected to the medical implant body and the electrode so as to electrolyze the medical implant body and form the thin film on the surface of the medical implant body. The thin film includes a surface, a plurality of walls and a plurality of paths. The walls are disposed on the surface, and formed to shapes of arc. The paths are also disposed on the surface, wherein the path is located among the walls. All of the walls and paths have a plurality of holes. The method for manufacturing the thin film of the medical implant of the present disclosure is adapted to form the thin film on the surface of the medical implant body by the chemical electrolysis machining process, whereby the manufacture cost can be decreased, the quality is stable, and the environmental pollution can be decreased.

### BRIEF DESCRIPTION OF THE DRAWINGS

The present disclosure will become more fully understood from the detailed description given herein below for illustration only, and thus are not limitative of the present disclosure, and wherein:

FIG. 1 is a sectional schematic view showing that a medical implant is implanted to a biological body according to an embodiment of the present disclosure;

FIG. 2A is a perspective view of a thin film of a medical implant according to an embodiment of the present disclosure;

FIG. 2B is an exploded top view of a thin film of a medical implant according to an embodiment of the present disclosure;

FIG. 2C is a perspective schematic view of a thin film of a medical implant according to an embodiment of the present disclosure;

FIG. 3 is a schematic view showing a device for manufacturing the thin film of the present disclosure;

FIG. 4 is an exploded top view of thin films according to a second embodiment of the present disclosure;

FIG. 5 is an exploded top view of thin films according to a third embodiment of the present disclosure;

FIG. 6 is an exploded top view of thin films according to a fourth embodiment of the present disclosure;

FIG. 7 is an exploded top view of thin films according to a fifth embodiment of the present disclosure; and

FIG. 8 is a chart showing test results of biological cyto-affinity of thin films of medical implants according to four embodiments of the present disclosure.

### DETAILED DESCRIPTION OF THE EMBODIMENT

In order to make the above features and advantages of the present disclosure more comprehensible, the present disclosure is illustrated below in detail with reference to the embodiments and the accompanying drawings.

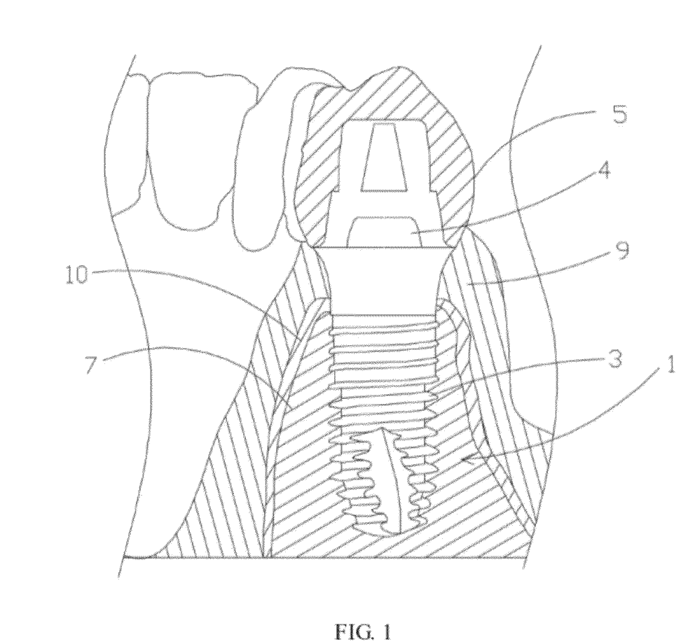

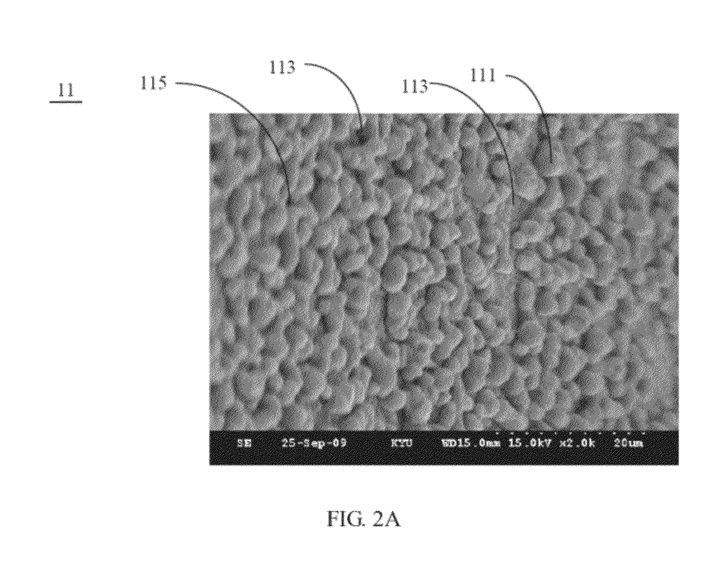

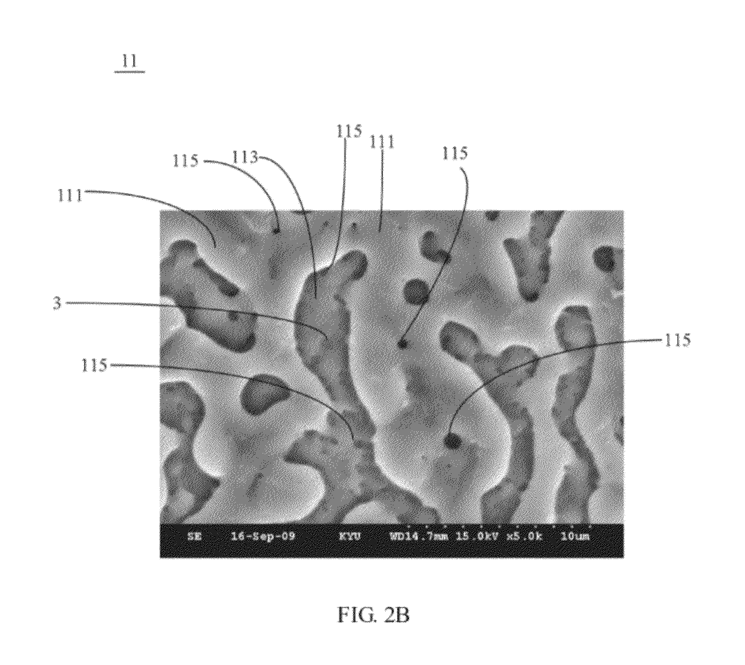

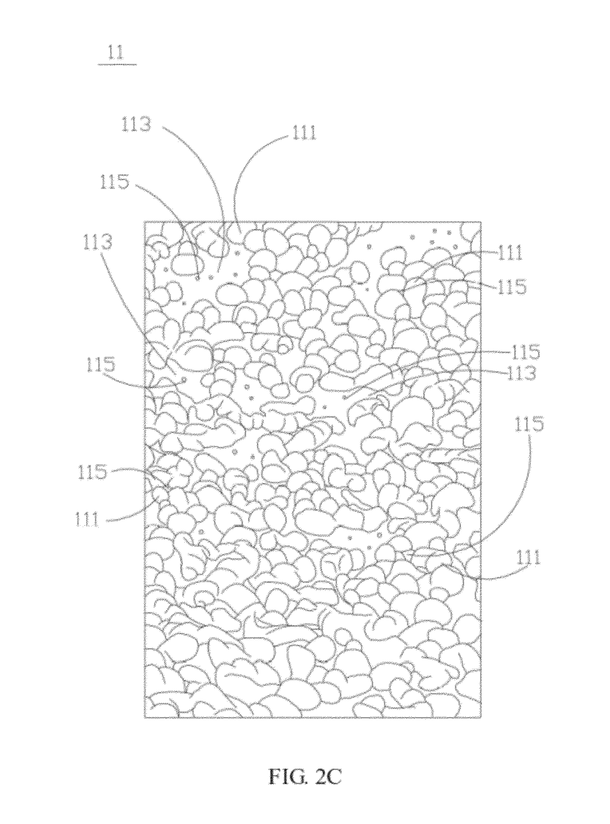

Please refer to FIG. 1, it depicts that a medical implant is implanted to a biological body according to an embodiment of the present disclosure. As shown in FIG. 1, the medical implant is an artificial tooth according to this embodiment. The body 1 of the artificial tooth has a dental implant 3, an abutment 4 and a false crown 5. The artificial tooth is implanted and mounted in an alveolar bone 7 of a human body or other animal body so as to replace a degenerative tooth or a damage tooth. When the dental implant 3 of the medical implant body 1 is implanted in the biological body, biological cells can grow slowly on the surface of the dental implant 3 of the medical implant body 1. For example, in this embodiment, cells of gingival 9, cortical bone 10 and alveolar bone 7 can grow slowly on the surface of the dental implant 3 of the medical implant body 1. In order to increase the biological cyto-compatibility and the biological cyto-affinity of the medical implant body 1 of the present disclosure, a thin film 11 shown in FIGS. 2A to 2C is formed on the surface of the dental implant 3 of the medical implant body 1. The thin film 11 includes a rough surface, whereby the biological cells can grow easily thereon so as to increases the biological cyto-compatibility and the biological cyto-affinity of the medical implant. The artificial tooth in this embodiment is merely the medical implant in one embodiment of the present disclosure for easily describing the present disclosure. The thin film 11 of the present disclosure can be formed on the surface of the various type medical implant, such as artificial joint, artificial bone screw and artificial bone plate, whereby the thin film 11 acts as a growable interface for biological cells.

Please refer to FIGS. 2A to 2C, they are a perspective view, an exploded top view and a perspective schematic view of a thin film of a medical implant according to an embodiment of the present disclosure. FIGS. 2A and 2B are photos of scanning electron microscope (SEM) of the thin film 11 of the present disclosure for clear describing the structure of the thin film 11 of the present disclosure. FIG. 2C is a perspective schematic view in accordance with the photo of the thin film 11 of the present disclosure. As shown in FIGS. 2A to 2C, the thin film 11 of the present disclosure includes a plurality of walls 111 and a plurality of paths 113. The walls 111 are protrusions, are formed to shapes of arc, have no acute anger, and are disposed on the surface of the thin film 11. Each of the paths 113 is located among the walls 111, and the paths 113 are also disposed on the surface of the thin film 11. In other words, the walls 111 and paths 113 are formed to the surface of the thin film 11. In addition, the walls 111 and paths 113 are irregular, and the walls 111 and paths 113 can have a plurality of holes 115. The holes 115 are micrometer level holes or sub-micrometer holes. In this embodiment of the present disclosure, the medical implant body 1 can be made of material of titanium (Ti) or titanium alloy, the thin film 11 can be made of titanium dioxide (TiO₂), the thickness of the thin film 11 is between 200 nm and 400 nm, and the diameter of the holes 115 is between 100 nm and 1000 nm.

The walls 111 of the thin film 11 of the present disclosure are protruded from the surface of the thin film 11, so the thickness of the walls 111 and paths 113 are different, and the thin film 11 has a three-dimensional perspective structure. The walls 111 are formed to shapes of arc (i.e. three-dimensional curved surface) and have no acute anger, whereby the biological cells can attach and grow on the thin film 11 very quickly. In addition, during the growth of the biological cells the holes 115 of the thin film 11 provide cell tissue, such as pseudopod, tentacle, etc. of the biological cells to attach and grow therein so as to increase biological cyto-compatibility and cyto-affinity of the thin film 11, whereby the thin film 11 acts as a good interface between the medical implant body 1 and the biological cells.

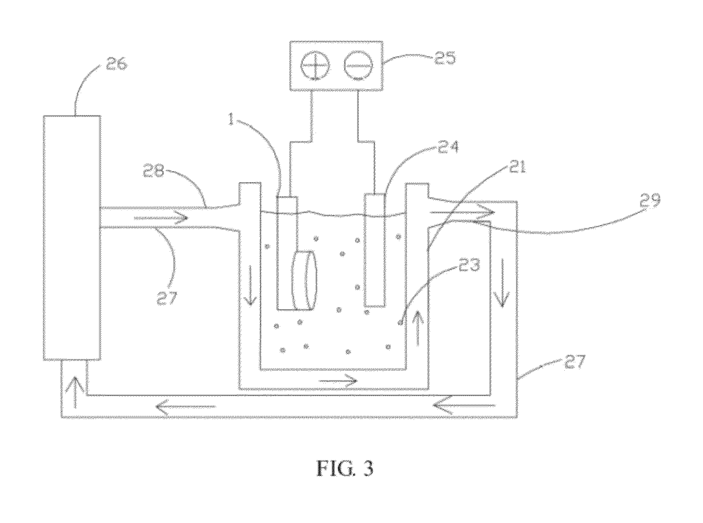

Please refer to FIG. 3, it is a schematic view showing a device for manufacturing the thin film of the present disclosure. As shown in FIG. 3, the device includes a tank 21 provided with an electrolyte 23 and an electrode 24. The medical implant body 1 is put in the tank 21 and dipped into the electrolyte 23. An anode of a power source unit 25 is electrically connected to the medical implant body 1, and a cathode of the power source unit 25 is electrically connected to the electrode 24. The power source unit 25 supplies power to the medical implant body 1 and the electrode 24 so as to execute a chemical electrolysis machining process, whereby the thin film 11 is formed on the surface of the medical implant body 1.

As described above, a method for manufacturing the thin film 11 of the present disclosure includes the following steps. A tank 21 having an electrolyte 23 and an electrode 24 is provided. The medical implant body 1 is put in the tank 21 and dipped into the electrolyte 23. An anode and a cathode of a power source unit 25 are electrically connected to the medical implant body 1 and the electrode 24 respectively so as to electrolyze the medical implant body 1 and form the thin film 11 on the surface of the medical implant body 1. Since the thin film 11 of the medical implant of the present disclosure is formed by the simple chemical electrolysis machining process, the manufacture process is simple and stable. Thus, the manufacture cost can be decreased, and the quality is stable and easily controlled. In addition, the electrolyte 23 is not strong acid or alkali, so the cost of waste water treatment and the environmental pollution can be decreased so as to have environmental protection.

In this embodiment, the electrode 24 of the present disclosure can be any conductor, such as metal, graphite, etc., and the electrolyte 23 can include SO₄²⁻, PO₄³⁻, H⁺, Na⁺, K⁺, Mg²⁺, Ca²⁺ or NH⁴⁺. For example, the concentration of SO₄²⁻ is adjusted to being more than and equal to 0 mole (M) and being less than 1.5 mole (M), and the concentration of PO₄³⁻ is controlled to being between 0.25 mole (M) and saturated concentration. When the concentration of SO₄²⁻ is equal to 0 mole (M), the electrolyte 23 only includes PO₄³⁻ whose the concentration is between 0.25 mole (M) and saturated concentration. In this embodiment, the PH value of the electrolyte 23 is between 3.5 and 9.5. The electrolyte 23 of the present disclosure has high concentration, and the power supplied by the power source unit 25 is low power, which has a power density being between 0.1 and 2.5 Amp/cm².

In addition, the device of the present disclosure further includes a temperature regulable unit 26 and two pipes 27. A side wall of the tank 21 is hollow, and has an inlet 28 and an outlet 29. The temperature regulable unit 26 is adapted to regulate the temperature of the electrolyte 23 in the tank 21. The temperature regulable unit 26 is communicated with the two pipes 27, and is physically connected to the inlet 28 and outlet 29 of the tank 21. The temperature regulable unit 26 transports a working medium (such as water) to the hollow side wall of the tank 21 through the pipe 27 and the inlet 28, and the working medium is returned to the temperature regulable unit 26 through the outlet 29 and the pipe 27 so as to control the temperature of the electrolyte 23 to a predetermined temperature. In this embodiment, the temperature of the electrolyte 23 is controlled by the working medium, so this method for controlling the temperature of the electrolyte 23 is an indirect temperature controlling method. However, in another embodiment of the present disclosure, another method for controlling the temperature of the electrolyte 23 can be a direct temperature controlling method. In other words, the circulation of the working medium (such as water) can be cancelled, the electrolyte 23 is directly inputted to and outputted from the temperature regulable unit 26 for heating or cooling the electrolyte 23 so as to also control the temperature of the electrolyte 23. In this embodiment of the present disclosure, the temperature of the electrolyte 23 is between 5 and 40 degrees.

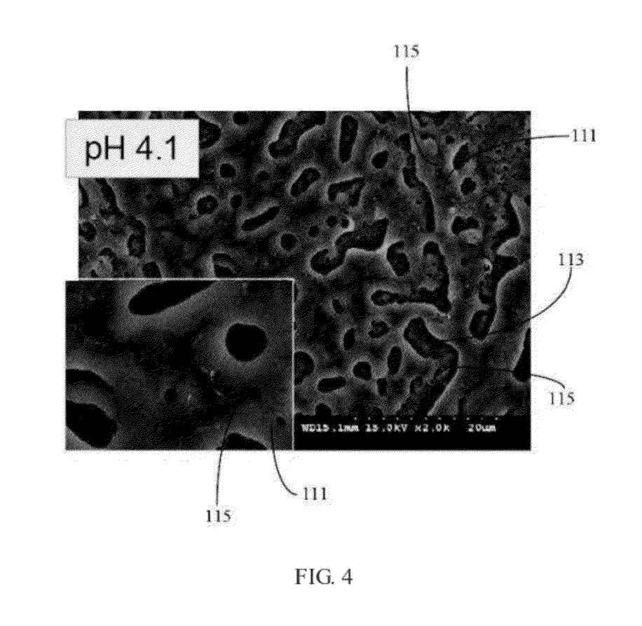

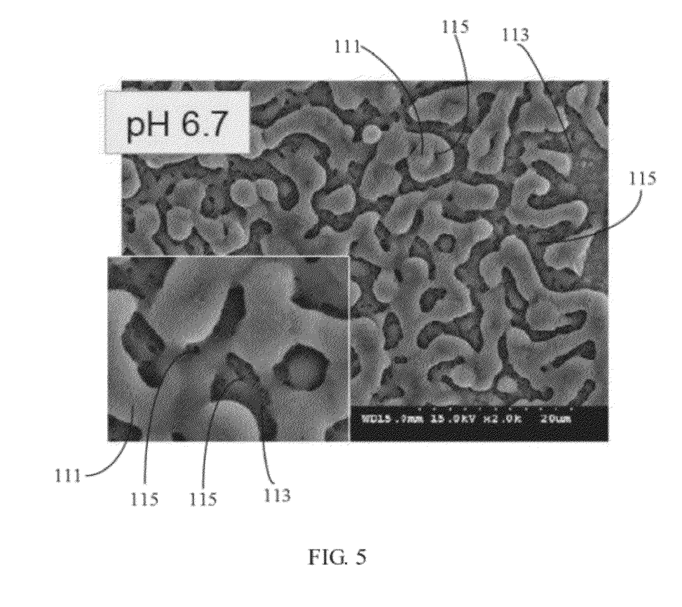

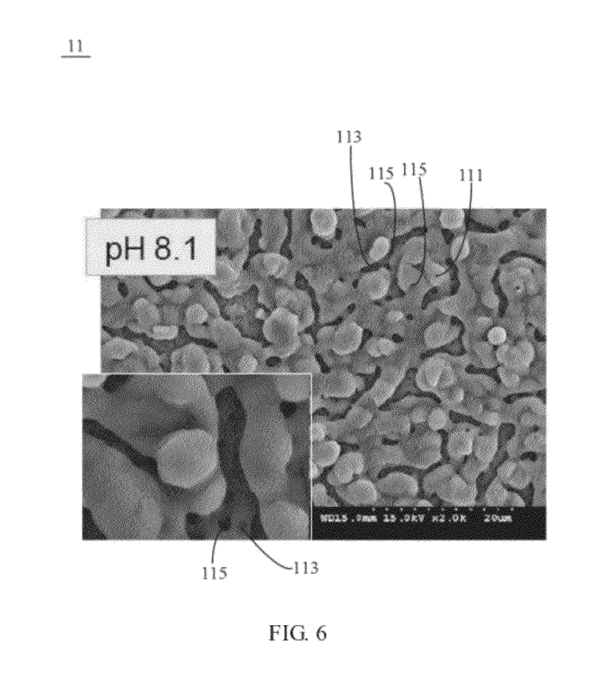

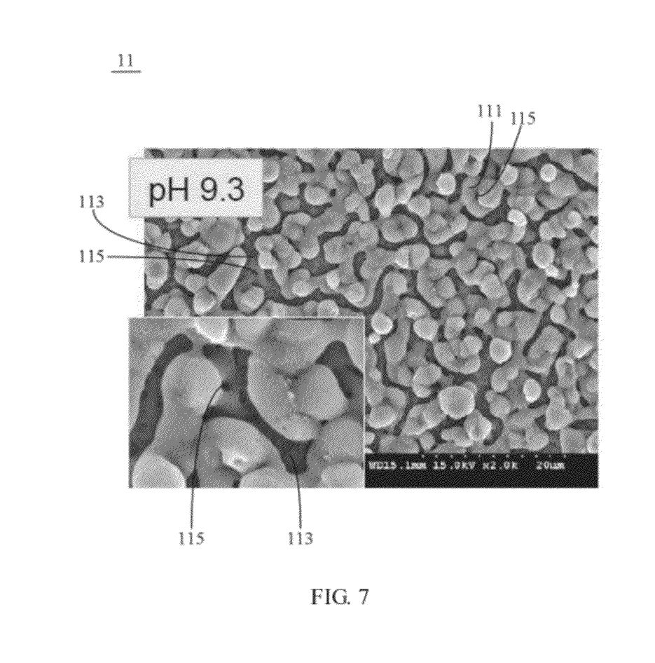

Please refer to FIGS. 4 to 7, they are exploded top views of thin films according to different embodiments of the present disclosure. In four embodiments, the medical implant bodies 1 are electrolyzed so as to form the thin films 11 in accordance with different four PH values, wherein the four PH values are 4.1, 6.7, 8.1 and 9.3. As shown in FIGS. 4 to 7, the medical implant can be formed with different structures of the thin films 11 in accordance with different conditions and of electrolysis. However, structures of the thin films 11 of the present disclosure have common features, for example, all of the thin films 11 include walls 111, paths 113 and holes 115, the walls 111 are formed to shapes of arc and have no acute anger, and the thickness of the walls 111 and paths 113 are different so as to have a three-dimensional perspective structure. Since the thin films 11 of the present disclosure include walls 111 and holes 115, whereby the biological cells can helpfully attach and grow on the thin film 11 quickly so as to increase biological cyto-compatibility and cyto-affinity of the medical implant.

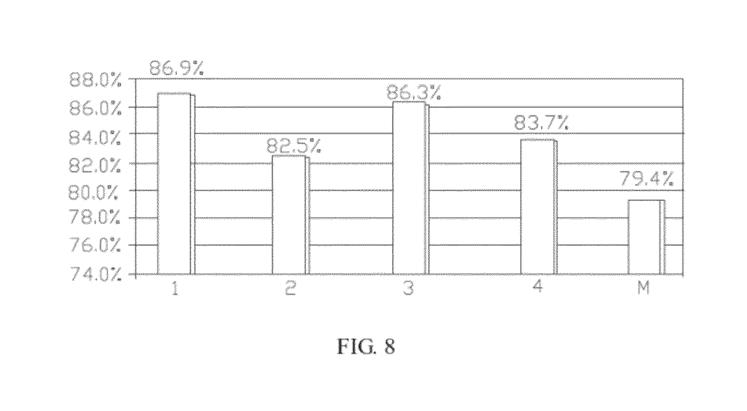

Please refer to FIG. 8, it is a chart showing test results of biological cyto-affinity of thin films of medical implants according to four embodiments of the present disclosure. The test results of biological cyto-affinity can show the reproduction status of the biological cells. The total value of activity and number of the biological cells can be calculated by measuring the metabolism concentration of the biological cells. FIG. 8 is finished by experiments in accordance with the thin films shown in FIGS. 4 to 7 and a smooth surface (M) without any surface treatment. As shown in FIG. 8, the thin films of the present disclosure have arc-shaped walls and holes, so test results of biological cyto-affinity are 86.9%, 82.5%, 86.3% and 83.7% respectively. All test results of biological cyto-affinity of the present disclosure are higher than test result (i.e. 79.4%) of the smooth surface (M) without any surface treatment. Thus, the biological cyto-compatibility and cyto-affinity of the thin film of the present disclosure is obviously better than those of the medical implant without any surface treatment.

In conclusion, the thin film of the medical implant of the present disclosure includes a plurality of walls and a plurality of paths. The walls are disposed on the surface of the thin film, are formed to shapes of arc, and have no acute anger. The path is located among the walls, and the walls are also disposed on the surface of the thin film. All of the walls and paths have a plurality of holes. According to the thin film of the present disclosure, the walls are formed to shapes of arc, and have no acute anger, whereby the biological cells can helpfully grow and attach on the thin film quickly. Furthermore, the thin film has the holes, which provide cell tissue, such as pseudopod, tentacle, etc. of the biological cells to grow and attach therein, whereby the biological cells helpfully grow and attach on the thin film so as to increase biological cyto-compatibility and cyto-affinity of the thin film. In addition, the method for manufacturing the thin film of the medical implant of the present disclosure is adapted to form the thin film on the surface of the medical implant body by the chemical electrolysis machining process. The chemical electrolysis machining process is simple, and thus the manufacture cost can be decreased, the quality is stable, and the environmental pollution can be decreased.

Although the present disclosure is disclosed above with reference to the above embodiments, the embodiments are not intended to limit the present disclosure. Equivalent replacements of variations and modifications made by any person skilled in the art without departing from the spirit and scope of the present disclosure still fall with the protection scope of the present disclosure.

What is claimed is:

1. A thin film of a medical implant, comprising:
a surface;
a plurality of walls disposed on the surface, and formed to shapes of arc; and
a plurality of paths disposed on the surface, wherein each of the paths is located among the walls;
wherein the walls and paths have a plurality of holes.

2. The thin film of the medical implant according to claim 1, wherein the medical implant includes a medical implant body, the thin film is located on a surface of the medical implant body, the medical implant body is made of material of titanium (Ti) or titanium alloy, and the thin film is made of titanium dioxide (TiO₂).

3. The thin film of the medical implant according to claim 1, wherein the diameter of the holes is between 100 nm and 1000 nm.

4. A method for manufacturing a thin film of a medical implant, comprising the following steps of:
providing a tank having an electrolyte and an electrode;
putting a medical implant body in the tank and dipping the medical implant body into the electrolyte;
electrically connecting an anode and a cathode of a power source unit to the medical implant body and the electrode respectively;
electrolyzing the medical implant body so as to form a thin film on the surface of the medical implant body, wherein the thin film comprises:
a surface;
a plurality of walls disposed on the surface, and formed to shapes of arc; and
a plurality of paths disposed on the surface, wherein each of the paths is located among the walls;
wherein the walls and paths have a plurality of holes.

5. The method according to claim 4, wherein the electrolyte includes SO₄²⁻, PO₄³⁻, H⁺, Na⁺, K⁺, Mg²⁺, Ca²⁺or NH⁴⁺.

6. The method according to claim 5, wherein the concentration of SO₄²⁻ is more than and equal to 0 mole (M) and is less than 1.5 mole (M), and the concentration of PO₄³⁻ is between 0.25 mole (M) and saturated concentration.

7. The method according to claim 4, wherein the PH value of the electrolyte is between 3.5 and 9.5.

8. The method according to claim 4, wherein the temperature of the electrolyte is between 5 and 40 degrees.

9. The method according to claim 4, wherein the medical implant body is made of material of titanium (Ti) or titanium alloy, and the thin film is made of titanium dioxide (TiO₂).

10. The method according to claim 4, wherein the diameter of the holes is between 100 nm and 1000 nm.

11. A medical implant, comprising:
a medical implant body;
a thin film disposed on the surface of the medical implant body, and comprising;
a surface;
a plurality of walls disposed on the surface, and formed to shapes of arc; and
a plurality of paths disposed on the surface, wherein each of the paths is located among the walls;
wherein the walls and paths have a plurality of holes.

12. The medical implant according to claim 11, wherein the medical implant body is made of material of titanium (Ti) or titanium alloy, and the thin film is made of titanium dioxide (TiO₂).

13. The medical implant according to claim 11, wherein the diameter of the holes is between 100 nm and 1000 nm.
